(19) United States
(12) Patent Application Publication
(10) Pub. No.: US 2026/0087781 A1
(43) Pub. Date: Mar. 26, 2026

(54) MEDICAL DIAGNOSIS ASSISTING DEVICE, MEDICAL DIAGNOSIS ASSISTING METHOD, AND SYSTEM

(71) Applicant: CASIO COMPUTER CO., LTD., Tokyo (JP)

(72) Inventor: Mitsuyasu NAKAJIMA, Tokyo (JP)

(73) Assignee: CASIO COMPUTER CO., LTD., Tokyo (JP)

(21) Appl. No.: 19/336,796

(22) Filed: Sep. 23, 2025

(30) Foreign Application Priority Data
Sep. 24, 2024 (JP) 2024-164738

(51) Int. Cl. G06V 10/764; A61B 5/00; G06T 7/00; G06V 10/774; G06V 10/776; G06V 10/82; G16H 50/20

(52) U.S. Cl. CPC G06V 10/764; A61B 5/7267; G06T 7/0012; G06V 10/776; G06V 10/82; G16H 50/20; G06T 2207/20081; G06T 2207/20084; G06T 2207/30096; G06V 10/774; G06V 2201/03

(57) ABSTRACT

A medical diagnosis assisting device includes one or more processors configured to classify a classification target in a medical image, using a classifier generated by multi-task learning based on (i) first information about benignity or malignancy or referral recommendation of the classification target in the medical image and (ii) second information about at least one of size, age, a body region, or a race of the classification target.

### CROSS-REFERENCE TO RELATED APPLICATIONS

This application claims the benefit of Japanese Patent Application No. 2024-164738, filed on Sep. 24, 2024, the entire disclosure of which is incorporated by reference herein.

### FIELD OF THE INVENTION

This application relates to a medical diagnosis assisting device, a medical diagnosis assisting method, and a system.

### BACKGROUND OF THE INVENTION

A technology that, using a classifier trained by machine learning, classifies a classification target in an image has been known. For example, Patent Literature 1 (Unexamined Japanese Patent Application Publication No. 2018-175226) discloses a medical image classification device that, using a determiner trained by a deep learning system, classifies medical images into a plurality of types of case areas.

### SUMMARY OF THE INVENTION

A medical diagnosis assisting device according to the present disclosure includes one or more processors configured to classify a classification target in a medical image, using a classifier generated by multi-task learning based on (i) first information about benignity or malignancy or referral recommendation of the classification target in the medical image and (ii) second information about at least one of size, age, a body region, or a race of the classification target.

### BRIEF DESCRIPTION OF DRAWINGS

A more complete understanding of this application can be obtained when the following detailed description is considered in conjunction with the following drawings, in which:

FIG. 1 is a block diagram illustrating a configuration of a classification device according to Embodiment 1;

FIG. 2 is a diagram illustrating inputting and outputting of a classifier in a training phase according to Embodiment 1;

FIG. 3 is a diagram illustrating a configuration of the classifier according to Embodiment 1;

FIG. 4 is a flowchart illustrating a flow of classifier generation processing that is executed by the classification device according to Embodiment 1;

FIG. 5 is a diagram illustrating inputting and outputting of the classifier in an inference phase according to Embodiment 1;

FIG. 6 is a flowchart illustrating a flow of classification processing that is executed by the classification device according to Embodiment 1;

FIG. 7A is a diagram illustrating a result of evaluation of the classifier according to Embodiment 1;

FIG. 7B is a diagram illustrating another result of the evaluation of the classifier according to Embodiment 1;

FIG. 7C is a diagram illustrating still another result of the evaluation of the classifier according to Embodiment 1;

FIG. 8 is a block diagram illustrating a configuration of a classifier generation device according to Embodiment 2; and

FIG. 9 is a block diagram illustrating a configuration of a classification device according to Embodiment 2.

### DETAILED DESCRIPTION OF THE INVENTION

Embodiments of the present disclosure are described below with reference to the drawings Note that the same or corresponding parts in the drawings are designated by the same reference numerals. A classification device 100 according to Embodiment 1 is a device that classifies benignity or malignancy of a classification target in an input image, using a classifier 30 generated by machine learning. In particular, the classification device 100 according to Embodiment 1 functions as a medical diagnosis assisting device that classifies, for a medical image in which a lesion is imaged as a classification target, whether the lesion is benign or malignant. As used herein, the medical image is an image imaged for the purpose of medical diagnosis, and is an image in which a region of a living body where a disease is suspected is imaged. A medical image is, as an example, an image obtained by imaging a skin lesion, such as a dermoscopy image. Alternatively, a medical image may be, without being limited to such an image, another type of image that can image a lesion, such as an endoscopic image, an X-ray image, a computed tomography (CT) image, and an ultrasonic image.

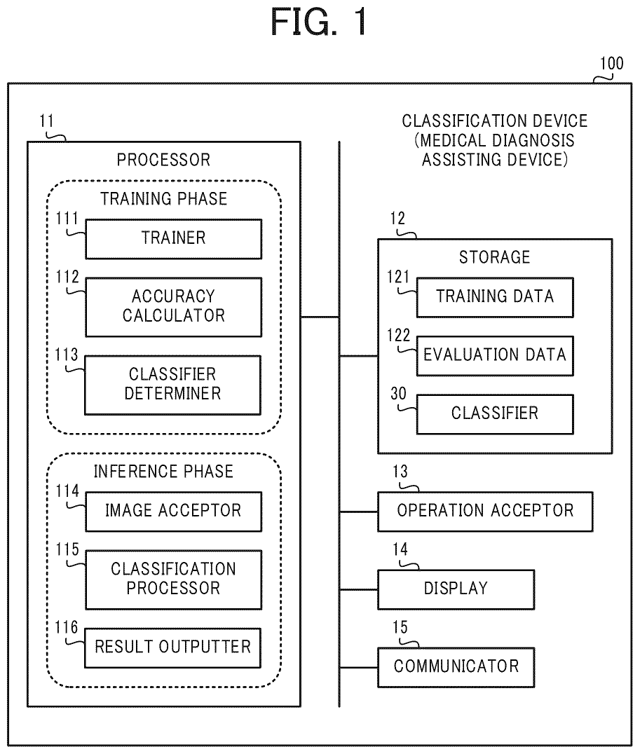

As illustrated in FIG. 1, the classification device 100 includes a processor 11, a storage 12, an operation accepter 13, a display 14, and a communicator 15. The processor 11 includes a central processing unit (CPU), a read only memory (ROM), and a random access memory (RAM). The CPU includes a microprocessor and the like, and is a central operation processor that executes various types of processing and operation. In the processor 11, the CPU retrieves a control program stored in the ROM and, using the RAM as a work memory, controls overall operation of the classification device 100. Note that processing performed by the processor 11 may be processing executed by only one CPU or processing executed by a plurality of CPUs.

The storage 12 is a nonvolatile memory, such as a flash memory and a hard disk. the storage 12 stores a program and data executed by the processor 11 as well as data generated by the processor 11. Specifically, the storage 12 stores the classifier 30, training data 121, and evaluation data 122. Details of the foregoing are described later.

The operation accepter 13 includes an input device, such as a keyboard, a mouse, and a touch panel, and accepts operation input from a user. The display 14 includes a display device, such as a liquid crystal display and an organic electro luminescence (EL) display, and displays various types of images under the control of the processor 11. The communicator 15 includes a communication interface to communicate with a device external to the classification device 100. For example, the communicator 15 communicates with an external device in conformance with a well-known communication standard, such as a local area network (LAN) and Universal Serial Bus (USB).

The processor 11 executes two phases of processing, namely a first phase that is a training phase of the classifier 30 and a second phase that is an inference phase performed by the classifier 30. The processor 11 includes, as functions in the training phase, a trainer 111, an accuracy calculator 112, and a classifier determiner 113. In addition, the processor 11 includes, as functions in the inference phase, an image accepter 114, a classification processor 115, and a result outputter 116. In the processor 11, the CPU functions as the above-described functional components by retrieving programs stored in the ROM into the RAM and executing the programs to perform control. Note that in the processor 11, a single CPU may function as the functional components in the training phase and the inference phase, or a plurality of CPUs may function as the functional components in the training phase and inference phase in cooperation with one another.

First, the training phase is described. The training phase is a phase in which a classifier 30 that is capable of accurately classifying input data is generated using a machine learning method. As used herein, the classifier 30 is a computer program for classifying benignity or malignancy of a classification target in an input image and is a trained model trained by machine learning using the training data 121.

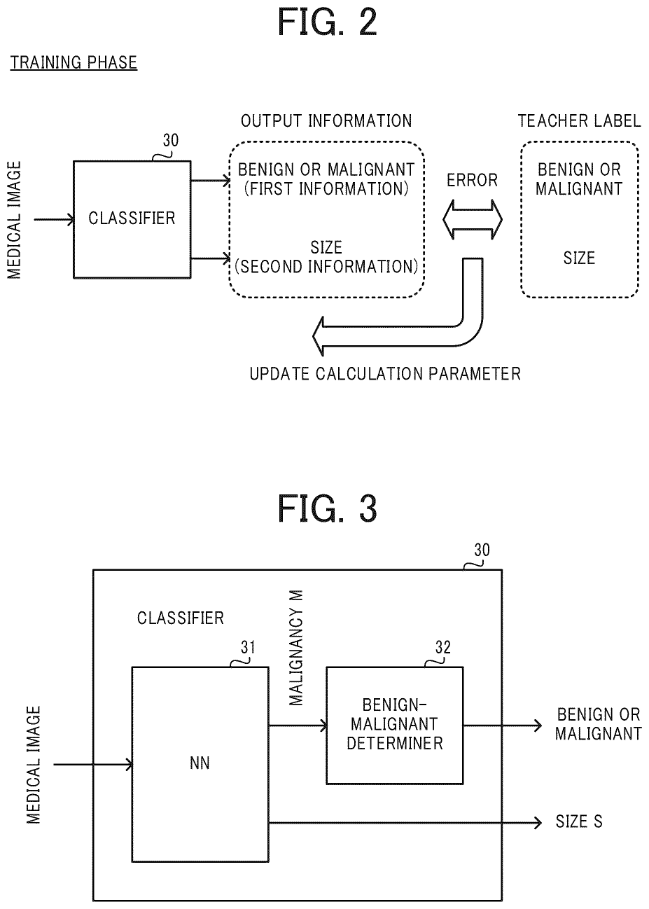

Specifically, as illustrated in FIG. 2, the classifier 30 accepts input of a medical image in which a lesion serving as a classification target is imaged. The classifier 30 outputs first information about benignity or malignancy of the lesion (attention region) imaged in the medical image and second information about a feature other than benignity or malignancy of the lesion, as output information for the input of the medical image. Specifically, the first information is information that indicates a result of classification of whether the lesion imaged in the medical image is benign or malignant. In addition, the second information is information that indicates an estimated value of size of the lesion in the medical image. As described above, the classifier 30 outputs, for input of one medical image, output information that indicates two results of classification, namely whether a lesion imaged in the medical image is benign or malignant and the size of the lesion.

More specifically, the classifier 30 includes, as illustrated in FIG. 3, a neural network (NN) 31 and a benign-malignant determiner 32. The NN 31 is a unit that executes main operation in the classifier 30. When specifically described, the NN 31 outputs malignancy M and size S for an input medical image, using a method such as logistic regression and deep neural network (DNN). As used herein, the malignancy M is a value indicating a probability that a lesion is malignant. The malignancy M has a value of 0 or more and 1 or less and means that the closer the malignancy M is to 0, the higher the probability that the lesion is benign is and the closer the malignancy M is to 1, the higher the probability that the lesion is malignant is. In addition, the size S is size of a lesion. As the size S, for example, diameter of a lesion is used.

As an example, the NN 31 is constructed by a neural network with a multi-layer structure, and has a plurality of layers including an input layer into which input data is input, an intermediate layer (hidden layer) that performs an operation, such as convolution and pooling, on the input data, and an output layer (fully-connected layer) that outputs a result of the operation. The NN 31 calculates malignancy M and size S of a lesion imaged in a medical image input to the input layer, in an intermediate layer, and outputs the calculated malignancy M and size S from the output layer.

The benign-malignant determiner 32 outputs first information indicating whether the lesion imaged in the input medical image is benign or malignant, based on the malignancy M output from the NN 31. When specifically described, the benign-malignant determiner 32 compares the malignancy M output from the NN 31 with a preset cutoff value. The benign-malignant determiner 32 determines the lesion to be malignant when the malignancy M is greater than the cutoff value, and determines the lesion to be benign when the malignancy M is less than the cutoff value. The cutoff value is set in advance to an appropriate value between 0 and 1 in such a way that the benign-malignant determiner 32 can appropriately determine benignity and malignancy.

The benign-malignant determiner 32 outputs such a determination result as the first information from the classifier 30. For example, the benign-malignant determiner 32 outputs value “1” as the first information when the lesion is determined to be malignant, and outputs value “0” as the first information when the lesion is determined to be benign. Note that the size S output from the NN 31 is output from the classifier 30 as it is as the second information.

Returning to FIG. 1, in the training phase, the trainer 111 performs machine learning using the training data 121. The training data 121 are a data set (data set for training) used by the trainer 111 to execute the machine learning. The training data 121 include a plurality of input images for training (hereinafter, referred to as “training image”) used as teacher data. Each of the plurality of training images is an image in which a lesion is imaged, and is an image where a correct answers to whether the imaged lesion is benign or malignant and the size of the lesion are known in advance. In the training data 121, to each training image, a correct malignancy LM and a correct size LS that serve as correct answers with respect to a lesion imaged in the training image are attached as teacher labels (correct labels) in advance. The correct malignancy LM is represented by a binary value of 1 or 0, and has 1 when the lesion is malignant and 0 when the lesion is benign. As the correct malignancy LM, a result of a pathological diagnosis (biopsy) can be used.

The trainer 111 performs multi-task learning, using the plurality of training images included in the training data 121 as teacher data and trains the classifier 30 to learn calculation parameters. As used herein, the multi-task learning is a machine learning method that trains one model by causing the model to learn a plurality of tasks simultaneously. In Embodiment 1, a plurality of tasks specifically corresponds to benign-malignant classification processing of classifying benignity or malignancy of a lesion, that is, whether the lesion is benign or malignant, imaged in a medical image and size estimation processing of estimating the size of the lesion. The trainer 111 causes a single classifier 30 to learn the above-described two tasks.

When specifically described, the trainer 111 inputs each of the plurality of training images included in the training data 121 to the classifier 30. In the classifier 30, the NN 31 calculates and outputs estimated values of the malignancy M and size S of a lesion imaged in the input training image. The trainer 111 adjusts calculation parameters of the classifier 30, using an error back-propagation method or the like in such a way that the malignancy M and the size S output from the NN 31 come close to correct malignancies LM and correct sizes LS attached to the input training images, respectively. The calculation parameters of the classifier 30 are, for example, weights of connections between layers in the neural network in the NN 31, that is, weights indicating connection strengths between a plurality of neurons (nodes). By adjusting the calculation parameters, estimated values of the malignancy M and the size S that the NN 31 outputs for the input training images change. The trainer 111 adjusts the calculation parameters by changing the calculation parameters in various manners in such a way that the malignancy M and the size S output from the NN 31 come close to the correct malignancy LM and the correct size LS to the extent possible, respectively. The trainer 111 optimizes the calculation parameters of the NN 31 by executing such adjustment processing of the calculation parameters for each of the plurality of training images included in the training data 121, and thereby constructs the neural network in the NN 31.

More specifically, the trainer 111 performs multi-task learning using a loss function E that is expressed by the following equation (1). The loss function E is a function for evaluating estimation error in estimation performed by the classifier 30. The loss function E is expressed by estimation error of the malignancy M, which is the first information, and estimation error of the size S, which is the second information. The estimation error of the malignancy M is calculated by a square of a difference (M-LM) between the malignancy M output from the NN 31 for input of a training image and a correct malignancy LM attached to the training image. Likewise, the estimation error of the size S is calculated by a square of a difference (S-LS) between the size S output from the NN 31 for input of a training image and a correct size LS attached to the training image. The trainer 111 calculates the loss function E by adding the above-described two estimation errors with weights using a hyperparameter α.

$$E = α \times {( M - LM )}^{2} + ( 1 - α ) \times {( S - LS )}^{2} \qquad ( 1 )$$

In the above-described equation (1), the hyperparameter α is a parameter that indicates weights of the estimation error of the malignancy M and the estimation error of the size S in the loss function E. The hyperparameter α is an external configuration variable that the user can freely set within a range of 0 or more and 1 or less. By changing the hyperparameter α, the weights of the two estimation errors can be adjusted. Specifically, since when the hyperparameter α is set larger, that is, brought close to 1, the weight of the estimation error of the malignancy M in the above-described equation (1) becomes larger, estimation accuracy of the malignancy M by the NN 31 becomes higher. In contrast, since when the hyperparameter α is set smaller, that is, brought close to 0, the weight of the estimation error of the size S in the above-described equation (1) becomes larger, estimation accuracy of the size S by the NN 31 becomes higher.

The hyperparameter α is set to a plurality of different values within a range from 0 to 1. The trainer 111 calculates the loss function E from the malignancy M and the size S, which are outputs of the NN 31, for each of cases where the hyperparameter α is changed to a plurality of values. The trainer 111 updates the calculation parameters of the NN 31 in such a way that the loss function E comes as close to 0 as possible, and employs the calculation parameters when the loss function E comes closest to 0 as the calculation parameters of the NN 31 at the set hyperparameter α. In this way, the trainer 111 performs the multi-task learning in each of a plurality of cases where the hyperparameter α is changed to a plurality of values, and thereby generates a plurality of candidates of the classifier 30.

Returning to FIG. 1, the accuracy calculator 112 calculates classification accuracy of the classifier 30 that has learned the calculation parameters of the NN 31 through the training performed by the trainer 111. As used herein, the classification accuracy of the classifier 30 is a value that represents a degree of to which extent the classifier 30 can correctly classify whether a lesion imaged in a medical image is benign or malignant. As an example, the accuracy calculator 112 calculates a correct diagnostic rate P(α) expressed by the following equation (2), as the classification accuracy. In the following equation (2), the number of correct benign estimations is the number of cases where a benign case, that is, a benign lesion, is correctly classified as benign, and the number of correct malignant estimations is the number of cases where a malignant case, that is, a malignant lesion, is correctly classified as malignant.

$$( 2 )$$

$$Correct diagnostic rate P ( α ) = ( ( number of correct benign estimations ) + ( number of correct malignant estimations ) ) / ( number of pieces of data )$$

The accuracy calculator 112 calculates the classification accuracy of the classifier 30, using the evaluation data 122. As used herein, the evaluation data 122 are a data set used to evaluate the classification accuracy of the classifier 30. The evaluation data 122 include a plurality of input images for evaluation (hereinafter, referred to as “evaluation images”). To each of the plurality of evaluation images, a correct malignancy LM and a correct size LS of an imaged lesion is attached as teacher labels, as with the training images. Note that all or some of the plurality of evaluation images in the evaluation data 122 may be the same as the training images. In addition, the machine learning by the trainer 111 and calculation of the classification accuracy by the accuracy calculator 112 may be performed using only the training data 121 by a cross validation method.

The accuracy calculator 112 inputs each evaluation image included in the evaluation data 122 to the classifier 30. The accuracy calculator 112 compares first information indicating benignity or malignancy that is output from the classifier 30 for input of each evaluation image with the correct malignancy LM of the evaluation image. The accuracy calculator 112 counts the number of cases where the first information indicates malignancy (1), that is, the number of cases where a lesion is correctly classified as malignant, for inputs of evaluation images the correct malignancies LM of which are malignant (1), as the “number of correct malignant estimations”. In addition, the accuracy calculator 112 counts the number of cases where the first information indicates benignity (0), that is, the number of cases where a lesion is correctly classified as benign, for inputs of evaluation images the correct malignancies LM of which are benign (0), as the “number of correct benign estimations”. The accuracy calculator 112 calculates the correct diagnostic rate P(α) in the above-described equation (2) by dividing a sum of the number of correct malignant estimations and the number of correct benign estimations by the number of evaluation images (the number of pieces of data) input to the classifier 30. The accuracy calculator 112 calculates the above-described correct diagnostic rate P(α) with respect to each of a plurality of candidates of the classifier 30 that is generated by the trainer 111 performing the multi-task learning with the hyperparameter α, which is used in the multi-task learning, changed to a plurality of values.

Returning to FIG. 1, the classifier determiner 113 determines, among the plurality of candidates of the classifier 30 generated by the trainer 111, a candidate the correct diagnostic rate P(α) of which calculated by the accuracy calculator 112 satisfies a predetermined criterion, as the classifier 30. When specifically described, the classifier determiner 113 calculates, with respect to each of the correct diagnostic rates P(α) calculated for a plurality of values of the hyperparameter α, a difference D of the correct diagnostic rate P(α) from a correct diagnostic rate P(1) of the classifier 30 in the case where the hyperparameter α is 1, using the following equation (3). The classifier determiner 113 determines a candidate for which a calculated difference Dis less than or equal to a standard value DS as a candidate the correct diagnostic rate P(α) of which satisfies the predetermined criterion, and determines the candidate as the classifier 30.

$$D = P ( 1 ) - P ( α ) \qquad ( 3 )$$

A case where the hyperparameter α is 1 is equivalent to a case where the weight of the estimation error of the size S in the loss function E is 0, that is, a case where the weight of the estimation error for the size S is the smallest. Therefore, the classifier determiner 113 calculates, with respect to each of the correct diagnostic rates P(α) of a plurality of candidates of the classifier 30 generated by changing the hyperparameter α to a plurality of values, a difference D between the correct diagnostic rate P(α) and a correct diagnostic rate P(1) of a candidate of the classifier 30 generated by the multi-task learning where the weight of the estimation error of the size S in the loss function E becomes zero. The classifier determiner 113 determines a candidate the difference D of which is less than or equal to the standard value DS as a candidate that satisfies the predetermined criterion. Note that the standard value DS is set to an extremely small value since the purpose is to detect a case not equal to the case where the hyperparameter α is equal 1.

Since the hyperparameter α is equivalent to the weight of the estimation error of the malignancy M in the loss function E, typically, the correct diagnostic rate P(1) in the case where the hyperparameter α is 1 is the largest, and the smaller the hyperparameter α becomes from 1, the lower the correct diagnostic rate P(α) becomes. Therefore, the criterion requiring that the difference D is less than or equal to the standard value DS is equivalent to that a degree of reduction in the classification accuracy from a classification accuracy when the hyperparameter α is 1 is small and falls within a predetermined range. Meanwhile, decreasing the hyperparameter α from 1 is equivalent to further improving the estimation accuracy of the size S in the multi-task learning and is considered to be equivalent to extracting and using characteristics more closely related to the size S.

The classifier determiner 113 determines a classifier 30 that, while increasing the estimation accuracy of the size S to the extent possible as described above, prevents the classification accuracy of benignity or malignancy by the classifier 30 from deteriorating to the extent possible, as the final classifier 30. For that purpose, the classifier determiner 113 determines, among candidates the correct diagnostic rate P(α) of which satisfies the predetermined criterion, a candidate that is generated with the hyperparameter α that maximizes the weight of the estimation error of the size S in the loss function E, that is, the hyperparameter α that is most distant from 1, as the classifier 30.

The reason why the estimation accuracy of the size S is increased in the training phase of the classifier 30 is to achieve stabilization (regularization) of the classifier 30. As used herein, the stabilization (regularization) means that a result of learning is optimized as a global optimum solution without falling into a local optimum solution. For example, a case is assumed where there are extremely few small-sized malignant cases in the training data 121. When the classifier 30 is trained, the calculation parameters of the NN 31 are learned in such a way that the loss function E becomes small to the extent possible. Since, in other words, the training is performed to improve the overall performance of the classifier 30, data of small-sized malignant cases that have a small number of samples are likely to be excluded. When the two features, namely the malignancy M and the size S, are independent, it is desirable to divide the training data 121 and generate a plurality of classifier 30 separately. However, when there is some correlation between the malignancy M and the size S, it is preferable to perform learning as a single classifier 30, using a large amount of data. In particular, in deep learning, there are some cases where even a feature that a person cannot recognize can be acquired. Therefore, it is expected that by performing learning with information about the size S incorporated, the classifier 30 actively uses a feature relating to the size S. As a result, data of small-sized malignant cases that have a small number of samples are expected to be actively made use of without being excluded. As described above, by sufficiently using the size information, the stabilization (regularization) of the classifier 30 can be achieved. In consideration of the above, the classifier determiner 113 determines a classifier 30 that is trained by the multi-task learning in such a way that the estimation accuracy of the size S becomes as high as possible within a range not causing the classification accuracy of benignity or malignancy to deteriorate largely, as the final classifier 30.

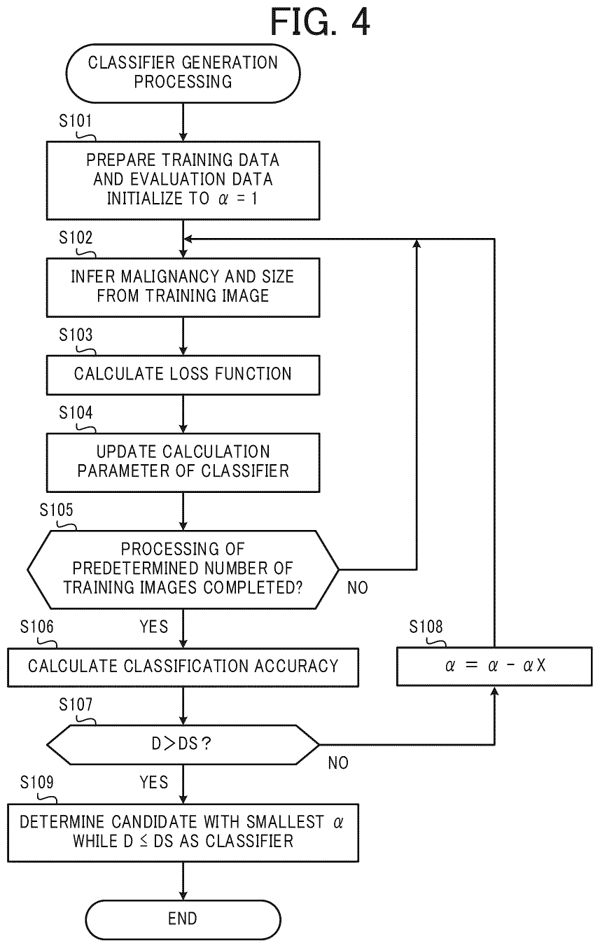

Next, with reference to FIG. 4, a flow of classifier generation processing executed by the classification device 100 in the training phase is described. The classifier generation processing illustrated in FIG. 4 is started when the operation accepter 13 accepts a start instruction from the user. The classifier generation processing illustrated in FIG. 4 is an example of a classifier generation method.

When the classifier generation processing is started, the processor 11 prepares the training data 121 and the evaluation data 122 and initializes the hyperparameter α in the loss function E to 1 (step S101). Next, the processor 11 selects a training image from a plurality of training images included in the training data 121. The processor 11 infers, from the selected training image, malignancy M and size S of a lesion imaged in the training image, using the classifier 30 (step S102). When specifically described, the processor 11 inputs a training image to the classifier 30 and acquires malignancy M and size S output from the classifier 30. Upon acquiring the malignancy M and the size S, the processor 11 calculates the loss function E, based on the obtained malignancy M and size S and a correct malignancy LM and a correct size LS attached to the training image, in accordance with the above-described equation (1) (step S103). Next, the processor 11 updates the calculation parameters of the classifier 30 in such a way that the loss function E comes close to 0 (step S104).

When the calculation parameters are updated, the processor 11 determines whether or not the processing in steps S102 to S104 has been executed using a predetermined number of training images (step S105). The predetermined number of training images may be all the training images included in the training data 121, or may be only some of all the training images included in the training data 121 as long as the number of the training images is a sufficient number to perform machine learning. When the processing using the predetermined number of training images is not completed (step S105; NO), the processor 11 returns the process to step S102. The processor 11 selects an unselected training image from the plurality of training images included in the training data 121, and repeats the processing in steps S102 to S105 on the newly selected training image. Through this processing, the processor 11 perform multi-task learning, using each of the plurality of training images included in the training data 121 in the case where the hyperparameter α is set to an initial value of 1. The processor 11 updates the calculation parameters of the classifier 30 in such a way that the loss function E comes close to 0 and generates a candidate of the classifier 30.

Subsequently, when the processing using the predetermined number of training images is completed (step S105; YES), the processor 11 functions as the accuracy calculator 112 and calculates classification accuracy of the classifier 30 (step S106). When specifically described, the processor 11 calculates a correct diagnostic rate P(α), using the above-described equation (2). When having calculated the classification accuracy, the processor 11 determines whether or not a difference D between the calculated classification accuracy and the classification accuracy of the classifier 30 when α=1 is greater than the standard value DS (step S107). Note that when the hyperparameter α is the initial value of 1, since the difference D is 0, the determination in step S107 results in NO.

When the difference D is less than or equal to the standard value DS (step S107; NO), the processor 11 sets a value obtained by reducing the current hyperparameter α by an amount obtained by multiplying the hyperparameter α by a ratio X as a new hyperparameter α (step S108). The ratio X is set in advance to a value such as 0.1, 0.05, or the like. When having set a new hyperparameter α, the processor 11 returns the process to step S102 and executes the processing in steps S102 to S107, using the new hyperparameter α. Through this processing, the processor 11 generates a candidate of the classifier 30 by performing the multi-task learning, using the new hyperparameter α and calculates the correct diagnostic rate P(α) of the generated candidate.

The processor 11 repeats the processing in steps S102 to S107 while gradually decreasing the hyperparameter α until the difference D between the newly calculated correct diagnostic rate P(α) and the correct diagnostic rate P(1) when α=1 becomes greater than the standard value DS. When finally the difference D becomes greater than the standard value DS (step S107; YES), the processor 11 terminates the update of the hyperparameter α. The processor 11 functions as the classifier determiner 113 and determine a candidate with the smallest hyperparameter α among a plurality of candidates the differences D of which is less than or equal to the standard value DS, as the classifier 30 (step S109). Consequently, the classifier generation processing illustrated in FIG. 4 terminates.

Returning to FIG. 1, second, the inference phase is described. The inference phase is a phase in which, using the classifier 30 that is generated in the training phase, whether an unknown lesion in an unknown medical image is benign or malignant is classified. In the inference phase, the image accepter 114 accepts input of an unknown medical image that serves as a classification target. As used herein, the unknown medical image is an image in which a lesion for which whether the legion is benign or malignant is unknown is imaged. The image accepter 114 accepts, in accordance with an instruction from the user accepted through the operation accepter 13, a specification of an unknown medical image serving as a classification target from a plurality of medical images stored in the storage 12 in advance. Alternatively, the image accepter 114 may accept an unknown medical image serving as a classification target from the outside by the communicator 15.

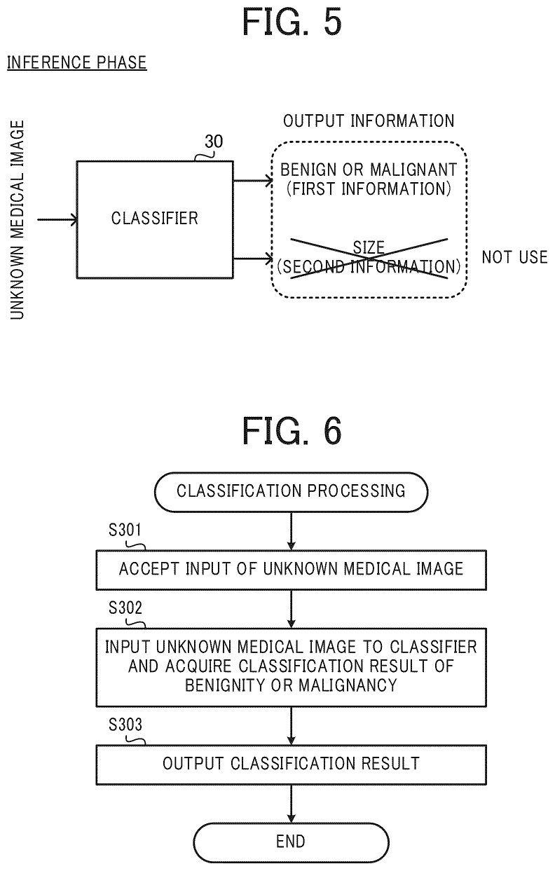

The classification processor 115, using the classifier 30 determined by the classifier determiner 113, classifies benignity or malignancy of a lesion that serves as a classification target in the unknown medical image accepted by the image accepter 114. Specifically, as illustrated in FIG. 5, the classification processor 115 inputs the unknown medical image accepted by the image accepter 114 to the classifier 30.

In the classifier 30, the NN 31 calculates malignancy M and size S of a lesion imaged in the input unknown medical image, using the calculation parameters on which the multi-task learning is performed by the trainer 111. The benign-malignant determiner 32 determines whether the lesion is benign or malignant by comparing the malignancy M calculated by the NN 31 with a cutoff value. The classifier 30 outputs first information indicating benignity or malignancy determined by the benign-malignant determiner 32 and second information indicating size S calculated by the NN 31. The classification processor 115 classifies whether the lesion is benign or malignant for the unknown medical image, based on the first information of the first information and second information output from the classifier 30, in this way. On this occasion, the classification processor 115 does not use the second information, that is, information about the size S, output from the classifier 30, in the inference phase.

The result outputter 116 outputs a result of classification performed by the classification processor 115. When specifically described, the result outputter 116 displays on the display 14 output information indicating whether the lesion in the unknown medical image is benign or malignant that is a result of classification performed by the classification processor 115. Alternatively, the result outputter 116 may output the output information by voice, or may output the output information to an external device via the communicator 15. Because of the output of information, the user can confirm a result of classification performed by the classification device 100.

Next, with reference to FIG. 6, a flow of classification processing executed by the classification device 100 in the inference phase is described. The classification processing illustrated in FIG. 6 starts when the operation accepter 13 accepts a start instruction from the user while the classifier 30 generated by the classifier generation processing illustrated in FIG. 4 is stored in the storage 12.

When the classification processing is started, the processor 11 functions as the image accepter 114 and accepts input of an unknown medical image in which an unknown lesion serving as a classification target is imaged (step S301). Next, the processor 11 functions as the classification processor 115 and inputs the unknown medical image to the classifier 30 and acquires a result of classification of benignity or malignancy indicated by the first information of the first information and second information output from the classifier 30 (step S302). Next, the processor 11 functions as the result outputter 116 and outputs output information indicating the acquired classification result (step S303). Consequently, the classification processing illustrated in FIG. 6 terminates. In the classification processing as described above, since the classifier 30 that is generated by the multi-task learning with respect to benignity or malignancy and size of a lesion in the classifier generation processing is used, it is possible to accurately classify benignity or malignancy of a lesion even when there is a bias in the training data 121 with respect to the sizes of lesions.

As described in the foregoing, the classification device 100 according to Embodiment 1 generates a classifier 30 that outputs, for input of a medical image, first information indicating whether a lesion imaged in the medical image is benign or malignant and second information indicating size of the lesion, by the multi-task learning and, using the generated classifier 30, classifies whether an unknown lesion imaged in an unknown medical image is benign or malignant. As described above, the classification device 100 according to Embodiment 1, to classify benignity or malignancy of a lesion, uses the classifier 30 on which the multi-task learning is performed in such a way as to output not only the first information about benignity or malignancy of the lesion but also the second information about the size of the lesion, which is a feature other than benignity or malignancy. As a result, even when collection of training images is not well balanced as to the sizes of lesions and there is a bias in the training data 121 with respect to the sizes of lesions, it is possible to obtain a stable classification result.

In particular, at clinical sites, many small-sized cases are benign diseases. Therefore, collection of a sufficient number of training images of small-sized malignant cases involves difficulty. When an imbalance in the distribution of diseases in the training data 121 occurs in this way, bias is likely to occur in the classification result. For example, when the number of malignant cases is smaller than the number of benign cases, a classification result is likely to be biased toward the benign side. In contrast, the classification device 100 according to Embodiment 1 generates the classifier 30, using size information of a lesion, which is not used in the inference phase, in the training phase. Therefore, since even when there is a significant bias in a case distribution in the training data 121, a learning result is optimized as a global optimum solution without falling into a local optimum solution, the stabilization (regularization) of the classifier 30 can be achieved. Therefore, a stable classification result can be obtained.

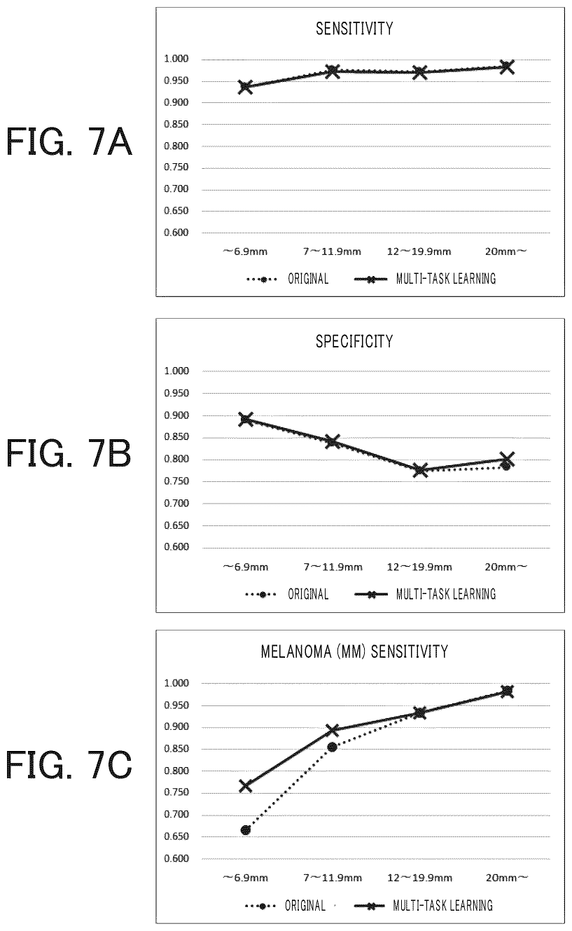

A result of evaluation in which the classifier 30 according to Embodiment 1 as described above is evaluated by experiment is illustrated in FIGS. 7A to 7C. The abscissas in FIGS. 7A to 7C represent size of a lesion imaged in a medical image. The ordinates in FIGS. 7A and 7B represent sensitivity and specificity of the classifier 30 that are evaluated without limiting the type of lesion, respectively. In addition, the ordinate in FIG. 7C represents sensitivity of the classifier 30 that is evaluated only when a lesion is melanoma. As used herein, the sensitivity is a ratio of cases where a malignant case is correctly classified as malignant, and the specificity is a ratio of cases where a benign case is correctly classified as benign. As an evaluation method, using a plurality of evaluation images divided by size of a lesion, sensitivity or specificity of a classifier 30 that is generated by the the multi-task learning, which is described in Embodiment 1, and sensitivity and specificity of a conventional classifier that is generated without performing the multi-task learning are evaluated by size. In FIGS. 7A to 7C, the solid lines indicate results of evaluation using the classifier 30 in Embodiment 1, and the dashed lines indicate results of evaluation using the conventional (original) classifier.

As a result, as illustrated in FIGS. 7A and 7B, when the type of lesion was not limited, there were no significant changes in sensitivity and specificity between the classifier 30 of Embodiment 1 and the conventional classifier, regardless of the sizes of lesions. On the other hand, in the case of melanoma illustrated in FIG. 7C, it was confirmed that while the sensitivity for small-sized lesions (less than 7 mm) was 0.667 when the conventional classifier was used, the sensitivity for small-sized lesions was improved to 0.767 when the classifier 30 of Embodiment 1 was used. Since melanoma is a dangerous malignant tumor, it is particularly important to classify the melanoma with high accuracy. Meanwhile, while melanoma is rare and there are few cases regardless of size, there are particularly few cases of small-sized melanoma. Therefore, collecting small-sized melanoma cases as the training data 121 involves difficulty. In contrast, it has been confirmed that when the classifier 30 in Embodiment 1 is used, small-sized melanoma, where there are fewer cases, can be classified with higher sensitivity than ever before without reducing classification accuracy for all types of lesions. By the experimental result described above, it is evident that the classifier 30 generated by the multi-task learning is effective in obtaining stable classification results even in the case where there is a bias in the case distribution in the training data 121.

Next, Embodiment 2 is described. Descriptions of the same constituent components and functions as those in Embodiment 1 are omitted. The classification device 100 according to Embodiment 1 described above includes, as illustrated in FIG. 1, both functions in the training phase and functions in the inference phase. In contrast, a classification device 100 according to Embodiment 2 does not include functions in a training phase, and a classifier generation device 200 that is a separate device from the classification device 100 includes the functions in the training phase.

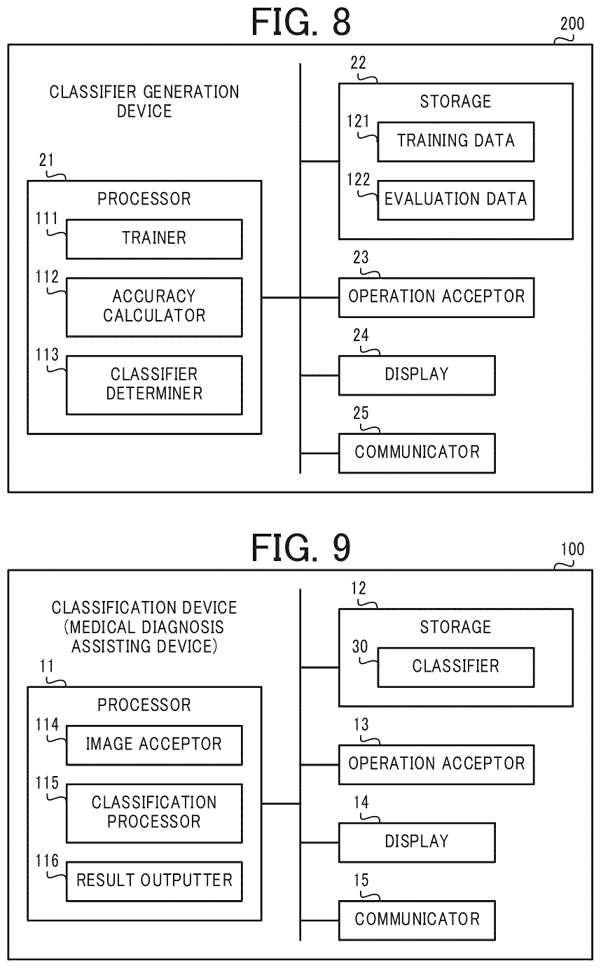

Specifically, as illustrated in FIG. 8, the classifier generation device 200 according to Embodiment 2 includes a processor 21, a storage 22, an operation accepter 23, a display 24, and a communicator 25. Since hardware configurations of the constituent components described above are the same as the processor 11, the storage 12, the operation accepter 13, the display 14, and the communicator 15 in the classification device 100, descriptions thereof are omitted. The processor 21 includes, as functions in the training phase, a trainer 111, an accuracy calculator 112, and a classifier determiner 113. In the processor 21, a CPU functions as the above-described functional components by retrieving programs stored in a ROM into a RAM and executing the programs to perform control. In addition, the storage 22 stores training data 121 and evaluation data 122 as data to be used in the training phase.

The respective functions of the processor 21 are the same as those in the trainer 111, the accuracy calculator 112, and the classifier determiner 113 included in the classification device 100 in Embodiment 1. When specifically described, the processor 21 executes classifier generation processing illustrated in FIG. 4 by the functions of the trainer 111, the accuracy calculator 112, and the classifier determiner 113. Through this processing, the processor 21 generates a classifier 30 on which multi-task learning is performed.

On the other hand, the classification device 100 according to Embodiment 2 has a configuration as illustrated in FIG. 9. In the classification device 100 according to Embodiment 2, although not including functions in the training phase, the processor 11 includes, as functions in the inference phase, an image accepter 114, a classification processor 115, and a result outputter 116. Functions of the constituent components described above are the same as those in Embodiment 1. The classification device 100 acquires the classifier 30 generated by the classifier generation device 200 from the classifier generation device 200 by means of, for example, communication via the communicator 15, and stores the acquired classifier 30 in the storage 12. The processor 11 executes, by the functions of the image accepter 114, the classification processor 115, and the result outputter 116, classification processing illustrated in FIG. 6, using the classifier 30 acquired from the classifier generation device 200. Through this processing, the processor 11 classifies whether a lesion imaged in a medical image is benign or malignant. As described above, in Embodiment 2, since the functions in the learning phase and the functions in the inference phase are performed by separate devices, it becomes possible to perform more flexible operation, such as utilizing the classifier 30 generated by the classifier generation device 200 in a plurality of classification devices 100.

In a conventional technology, there are some cases where, depending on a classification target, it is difficult to collect training data for machine learning in a balanced manner. In such a case, since bias occurs in the training data, bias is liable to occur in a classification result. According to the present disclosure, it is possible to obtain stable classification results even when there is a bias in the training data. Although the embodiments of the present disclosure are described above, the above-described embodiments are only examples, and the scope of application of the present disclosure is not limited to the embodiments. That is, various applications of the embodiments of the present disclosure are possible, and all embodiments are included in the scope of the present disclosure.

For example, in the above-described embodiments, the accuracy calculator 112 calculates a correct diagnostic rate P(α) expressed by the equation (2), as classification accuracy. However, the accuracy calculator 112 may calculate, without being limited to the correct diagnostic rate, another index value as the classification accuracy. For example, the accuracy calculator 112 may calculate an F1 score that is a harmonic average of sensitivity and precision, as the classification accuracy. As used herein, the sensitivity is a ratio of cases where a malignant case is correctly classified as malignant. The sensitivity is obtained by calculating a ratio of the number of pieces of first information from the classifier 30 indicating malignancy (1) to the number of inputs of a plurality of evaluation images the correct malignancies LM of which indicate malignancy (1). In addition, the precision is a ratio of actually malignant cases among data classified as malignant. The precision is obtained by calculating a ratio of the number of evaluation images the correct malignancies LM of which are malignant (1) among a plurality of evaluation images for which the first information from the classifier 30 indicates malignancy (1). In addition, in the above-described embodiments, the trainer 111 evaluates estimation error of the first information and second information, using the loss function E expressed by the equation (1). However, the trainer 111 may evaluate the estimation error of the first information and second information, using, without being limited to the loss function E, error represented by cross-entropy.

In the above-described embodiments, the second information that is used in the multi-task learning and is information about a feature other than benignity or malignancy of a classification target is information about size of a lesion. However, the second information may be information other than size and may, for example, be information indicating age, a body region, a race, or the like of a classification target. As used herein, the age of a classification target refers to age of a patient who has a lesion serving as a classification target. In general, there are fewer malignant cases at a young age, as with fewer malignant cases of small size.

Therefore, by performing the multi-task learning using age information as the second information, it is possible to obtain stable classification results even when there is a bias in the training data 121 with respect to age, as with the above-described embodiments. In addition, the body region of a classification target refers to a region of the body where a lesion serving as a classification target exists (for example, the facial region, the palmoplantar region, the mucosal region, or the like). The race of a classification target refers to a race of a patient who has a lesion serving as a classification target (such as the white race, the black race, and the yellow race). Since there are also some cases where collecting training images in a balanced manner involves difficulty, depending on a body region or a race, by performing the multi-task learning using such information as the second information, stable classification results can be obtained even when there is a bias in the training data 121. As described above, there is an advantageous effect that using information that tends to cause a bias in the training data 121 as the second information enables stable classification results to be obtained.

In the above-described embodiments, the classification device 100 classifies benignity or malignancy of a lesion. However, the classification device 100, without being limited to the configuration, may be a device that classifies a referral recommendation of a classification target. As used herein, the referral recommendation means recommending a patient having a lesion serving as a classification target to be referred to another hospital. For example, it is conceivable to refer a patient to a large-scale hospital capable of performing more specialized examinations from a small clinic. While in the above-described embodiments, the correct malignancy LM indicating malignant (1) or benign (0) and the correct size LS are attached to each training image as teacher labels, in a case where the classification device 100 classifies a referral recommendation, information indicating whether referral recommendation is required (1) or not required (0) is attached to each training image as a teacher label in place of the correct malignancy LM. The trainer 111 performs the multi-task learning, using such training data 121. Because of this configuration, the trainer 111 generates a classifier 30 that outputs first information indicating whether or not a referral recommendation is required for a lesion imaged in a medical image (existence or nonexistence of a referral recommendation) and second information relating to a feature other than benignity or malignancy of the lesion for input of the medical image. Alternatively, by, while keeping using the correct malignancy LM as the teacher label, setting a cutoff value used by the benign-malignant determiner 32 higher than in a case of classifying benignity or malignancy of a lesion, the classification device 100 may be used as a referral recommendation classifier.

In the above-described embodiments, the classification device 100 is a medical diagnosis assisting device that classifies whether a lesion imaged in a medical image is benign or malignant. However, the classification device 100 is not limited to serving as a medical diagnosis assisting device. For example, the classification device 100 may be an inspection device that accepts input of an inspection image in which a construction, such as a building, a road, and a bridge, is imaged and that classifies, as quality of the construction, whether there occurs an abnormality in the construction, based on cracks, front surface shape, and the like of the construction imaged in the inspection image. In this case, the classification target is not equivalent to a lesion imaged in a medical image, but a construction imaged in an inspection image. The quality of the classification target is not equivalent to benignity or malignancy of the lesion, but presence or absence of an abnormality in the construction.

In addition, the classification device 100, without being limited to outputting binary information as described above, such as the quality of a lesion (benign or malignant), whether or not a referred diagnosis is required (the presence or absence of referred diagnosis), and the quality of a construction (presence or absence of abnormality), as a classification result, may output information exceeding binary values as a classification result. For example, the classification device 100 may, using a classifier 30 capable of performing eight disease classification, output a classification result indicating which of the eight diseases a lesion corresponds to. As used herein, the eight diseases refer to, as an example, eight major diseases, namely melanoma, basal cell carcinoma, other malignant diseases, pigmented nevus, seborrheic keratosis, dermatofibroma, hemangioma, and other benign diseases. In this case, the NN 31 outputs probability values each of which indicates a probability that a lesion imaged in an input medical image corresponds to one of the eight diseases, in place of the malignancy M in the above-described embodiments. For example, when the input medical image is a melanoma image, the NN 31 is trained by the trainer 111 in such a way that a probability value corresponding to melanoma comes close to 1 and probability values corresponding to the other seven diseases come close to 0. The benign-malignant determiner 32 determines that a disease with the highest probability value among the eight probability values output from the NN 31 is a disease corresponding to the lesion imaged in the input medical image. For example, when the probability value of melanoma is the highest, the benign-malignant determiner 32 determines that the lesion is melanoma. The classifier 30 outputs a determination result determined by the benign-malignant determiner 32 as described above, as first information. In the classification device 100, the classification processor 115 classifies which of the eight diseases the lesion imaged in the input medical image corresponds to, based on the first information output from the classifier 30, and the result outputter 116 outputs the classification result.

In the above-described embodiments, the processor 11 or 21 functions as respective constituent components illustrated in FIG. 1, 8, or 9 by the CPU executing programs stored in the ROM or the storage 12. However, the processors 11 and 21 may be dedicated hardware. The dedicated hardware is, for example, a single circuit, a composite circuit, a programmed processor, an application specific integrated circuit (ASIC), a field-programmable gate array (FPGA), or a combination of the foregoing. When the processors 11 and 21 are dedicated hardware, each of the functions of the constituent components may be achieved by an individual piece of hardware, or the functions of the constituent components may be collectively achieved by a single piece of hardware. In addition, among the functions of the constituent components, some functions may be achieved by dedicated hardware and the other functions may be achieved by software or firmware. As described above, the processors 11 and 21 can achieve the above-described functions by hardware, software, firmware, or a combination of the foregoing.

By applying a program that defines the operation of the above-described classification device 100 or classifier generation device 200 to a computer, such as a personal computer and a cloud server, it is possible to cause the computer to function as the above-described classification device 100 or classifier generation device 200. In addition, a method for distributing such a program is arbitrarily determined, and the program may be distributed stored in a non-transitory computer-readable recording medium, such as a compact disk ROM (CD-ROM), a digital versatile disk (DVD), a magneto optical disk (MO), and a memory card, or may be distributed via a communication network, such as the Internet. In addition, the above-described classification device 100 or classifier generation device 200 may be a system including a server and a device.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

1. A medical diagnosis assisting device, comprising
one or more processors configured to classify a classification target in a medical image, using a classifier generated by multi-task learning based on (i) first information about benignity or malignancy or referral recommendation of the classification target in the medical image and (ii) second information about at least one of size, age, a body region, or a race of the classification target.

2. The medical diagnosis assisting device according to claim 1, wherein the one or more processors classify, using the classifier generated by the multi-task learning based on the first information about benignity or malignancy of the classification target in the medical image and the second information, benignity or malignancy of the classification target in the medical image.

3. The medical diagnosis assisting device according to claim 1, wherein the one or more processors classify, using the classifier generated by the multi-task learning based on the first information about referral recommendation of the classification target in the medical image and the second information, referral recommendation of the classification target in the medical image.

4. The medical diagnosis assisting device according to claim 1,
wherein a plurality of candidates of the classifier is generated by performing the multi-task learning with the hyperparameter, the hyperparameter being used in the multi-task learning, changed to a plurality of values,
classification accuracy of each of the generated plurality of candidates is calculated, and
the classifier is, among the plurality of candidates, a candidate the calculated classification accuracy of which satisfies a predetermined criterion.

5. The medical diagnosis assisting device according to claim 4,
wherein the multi-task learning is performed, using a loss function that is represented by estimation error of the first information and estimation error of the second information, and
the hyperparameter is a parameter that indicates weights of estimation error of the first information and estimation error of the second information in the loss function.

6. The medical diagnosis assisting device according to claim 5,
wherein the predetermined criterion is satisfied in a case where a difference between the calculated classification accuracy and classification accuracy of a candidate that is generated with a hyperparameter that minimizes a weight of estimation error of the second information in the loss function among the plurality of candidates is less than or equal to a standard value, and
the classifier is, among candidates the classification accuracies of which satisfy the predetermined criterion, a candidate that is generated with a hyperparameter that maximizes a weight of estimation error of the second information in the loss function.

7. The medical diagnosis assisting device according to claim 1, wherein the classifier does not use the second information as output information in a case in which the classifier classifies the classification target in the medical image.

8. A medical diagnosis assisting method, comprising
classifying a classification target in a medical image, using a classifier generated by multi-task learning based on (i) first information about benignity or malignancy or referral recommendation of the classification target in the medical image and (ii) second information about at least one of size, age, a body region, or a race of the classification target.

9. A system including a server and a device, the system comprising
one or more processors,
wherein the one or more processors classify a classification target in a medical image, using a classifier generated by multi-task learning based on (i) first information about benignity or malignancy or referral recommendation of the classification target in the medical image and (ii) second information about at least one of size, age, a body region, or a race of the classification target.
